(19) United States
(12) Patent Application Publication
(10) Pub. No.: US 2025/0387338 A1
(43) Pub. Date: Dec. 25, 2025

(54) FILM FORMING DELAYED/ENTERIC FORMULATIONS FOR HARD SHELL CAPSULES, FILMS AND COATINGS

(71) Applicant: NUTRITION & BIOSCIENCES USA 1, LLC, Wilmington, DE (US)

(72) Inventors: Jin ZHAO, Midland, MI (US); Stephanie ROBART, Midland, MI (US)

(21) Appl. No.: 18/877,363

(22) PCT Filed: Jun. 19, 2023
(86) PCT No.: PCT/US2023/068667

§ 371 Date: Dec. 20, 2024

Related U.S. Application Data
(60) Provisional application No. 63/353,862, filed on Jun. 21, 2022.

(51) Int. Cl. A61K 9/48

(52) U.S. Cl. CPC A61K 9/4816; A61K 9/4866; A61K 9/4891

(57) ABSTRACT

The present invention is directed to aqueous film-forming compositions suitable for capsule films, capsule shells, as wells as methods for producing such capsule film, capsule shell and the use thereof.

### FIELD OF THE INVENTION

The present invention is directed to aqueous film-forming compositions suitable for capsule films, capsule shells, as wells as methods for producing such capsule film, capsule shell and the use thereof.

### BACKGROUND OF THE INVENTION

Capsules are widely used in administration of pharmaceuticals and nutritional to humans and animals. Capsules also have divergent uses, such as serving as reservoirs of plant fertilizer for easy application, of colorants, of food materials or food supplements, and of cosmetic ingredients. Alginate is a convenient material for the film part of the capsule because it can be formed in the process of preparing the capsule.

Alginates are high molecular weight polysaccharides extracted with dilute alkali from various species of brown seaweeds. They are hydrophilic colloidal carbohydrates that are water-soluble biopolymers of colloidal nature when hydrated. In terms of chemical structure, they are linear copolymers of 1,4 linked β-D mannuronic acid and β-L-guluronic acid that consist of three distinct polymer segments: Polymannuronic acid segments (M blocks), polyguluronic acid segments (G blocks), and alternating mannuronic acid and guluronic acid units (MG blocks). A wide range of these polymers are offered as alginic acid, various salt forms, and propylene glycol esters that vary in molecular weight, particle size, M/G ratio, and viscosity. Alginate is of interest as a potential biopolymer film or coating component because of its unique colloidal properties, which include thickening, stabilizing, suspending, film forming, gel producing, and emulsion stabilizing. Alginic acid and its derivatives have been used in food processing industry, in biotechnology industry for producing beads for immobilization of cells or enzymes, in pharmaceutical industry for tablet disintegration, controlled release, encapsulation, as films and coatings, lubricating agents, prevention of gastric reflux, gelling and as thickening agents to stabilize emulsions and suspensions.

Sodium alginate is a natural, biocompatible, biodegradable and hydrophilic polymer suitable for the entrapment of bioactive ingredients, for example, drugs. Sodium alginate hydrates readily in water, the solutions are stable in the pH range of 4-10 and upon drying form strong transparent films.

U.S. Pat. No. 4,365,060 discloses enterosoluble capsules which are said to have excellent enterosolubility behavior. The capsules are prepared from a mixed ester of an alkyl-, hydroxyalkyl- or hydroxyalkal alkylcellulose esterified with succinyl anhydride and an aliphatic monocarboxylic acid anhydride. The U.S. patent discloses that the cellulose derivative can be shaped into capsules not only by the conventional dipping method but also by the plastic deformation at an elevated temperature under pressure such as compression molding, vacuum forming, matched-mold forming and the like. The U.S. patent states that the enterosoluble capsules have excellent pliability, even with addition of no or a very small amount of plasticizer. Unfortunately, the dipping method requires the use of an organic solvent for dissolving the mixed ester of an alkyl-, hydroxyalkyl- or hydroxyalkal alkylcellulose. Organic solvents are often not desirable for pharmaceutical or nutritional uses. Moreover, the handling of organic solvents adds to the complexity of the process for producing the capsules. Forming capsules by plastic deformation is often not desirable either due to the significant thermal stress and thermal degradation caused by the heat that is needed for thermoforming and the quite complex and expensive molds that are needed for thermoforming processes.

U.S. Pat. No. 9,433,585 describe compositions for manufacturing enteric soft capsules comprising gelatin, methyacrylic copolymers, one or more plasticizers, an alkaline aqueous solvent and, optionally, other ingredients such as colors, and flavors. Capsules made from such compositions are useful for delivery of acid-sensitive dosage forms to the lower gastrointestinal tract or to provide delayed release of active pharmaceutical ingredients.

US2012/0161364 describe an aqueous composition for an enteric hard capsule, a method of preparing an enteric hard capsule, and an enteric hard capsule prepared using the method.

WO2013/164121 describe an aqueous composition comprising hydroxypropyl methyl cellulose acetate succinate (HPMCAS) polymer dispersed in water, wherein the dispersed polymer is partially neutralized with at least one alkaline material. This disclosure also relates to compositions for use in methods of making capsule shells endowed with bulk enteric properties.

However, the enteric polymer used in the above-mentioned prior art are not affirmed as generally recognized as safe (GRAS) for food and nutritional applications. Considering the growing demand of encapsulating probiotics and other actives that are preferred to be delivered in the intestinal tract, there is still a need for capsules materials that are GRAS and subsequent capsule films suitable for delayed release and/or enteric use.

### BRIEF DESCRIPTION OF THE DRAWINGS

FIG. 1. Rheology of inventive formulations of those listed in Table 1 and 2.

FIG. 2. Film puncture strength of inventive formulations of those listed in Table 1, 2 and 3 which are sufficient for capsule forming.

FIG. 3. An example of film appearance after 2 hrs in pH 1.2 dissolution medium for films made from formulations from Table 1, 2 and 3.

### SUMMARY OF THE INVENTION

It is an object of embodiments of the invention to provide aqueous film-forming composition suitable for improved capsule coatings, films or capsule shells.

The present invention relates in a broad aspect to aqueous film-forming compositions suitable for delayed release or enteric release of pharmaceutically or nutraceutically active ingredients.

Accordingly, in a first aspect the present invention relates to an aqueous film-forming composition comprising a) alginate having a viscosity in the range of about 2 to about 200 mPaS (1 mPaS is equivalent to 1 centipoise, also be referred to as cp, cps or cPs) measured at 1 wt % solution in water at 20° C. and b) a film forming polymer having a viscosity in the range of about 2 to about 20 mPaS measured at 2 wt % solution in water at 20° C.

In a second aspect the present invention relates to the use of a composition according to the invention for encapsulation of a drug, a nutritional or food supplement, or a combination thereof.

In a third aspect the present invention relates to a capsule film made from the composition according to the invention.

In a further aspect the present invention relates to a capsule shell comprising a) alginate having a viscosity in the range of about 2 to about 200 mPaS measured at 1 wt % solution in water at 20° C. and b) a film forming polymer having a viscosity in the range of about 2 to about 20 mPas measured at 2 wt % solution in water at 20° C.

In a further aspect the present invention relates to a capsule comprising a capsule shell prepared from the composition according to the invention.

In a further aspect the present invention relates to a process for producing capsule film, capsule shell, or capsule comprising the steps of providing the aqueous composition according to the invention, pre-heating moulding pins to a temperature at highest viscosity point ±5 C of the aqueous composition above the gelation temperature, dipping the pre-heated moulding pins into the aqueous composition at a temperature below gelation temperature, forming a film on said moulding pins by withdrawing said pins from said composition, and drying the film on the moulding pins above the gelation temperature.

### DETAILED DESCRIPTION OF THE INVENTION

As detailed above the present invention relates to an aqueous film-forming composition comprising a) alginate having a viscosity in the range of about 2 to about 200 mPaS measured at 1 wt % solution in water at 20° C. and b) a film forming polymer having a viscosity in the range of about 2 to about 20 mPaS measured at 2 wt % solution in water at 20° C.

In some embodiments the alginate is selected from the group consisting of sodium alginate, potassium alginate, and other monovalent alginate salts, and mixtures thereof.

In some embodiments wherein the alginate is sodium alginate.

In some embodiments the alginate has a viscosity in the range of about 2 to about 100 mPaS, more preferred in the range of about 2 to about 30 mPaS measured at 1 wt % solution in water at 20° C.

In some embodiments the film forming polymer is selected from the group consisting of hydroxypropyl methylcellulose (HPMC), carboxymethyl cellulose (CMC) and methylcellulose (MC), hydroxypropyl cellulose (HPC); and mixtures thereof.

In some embodiments the film forming polymer has a viscosity in the range of about 3 to about 15 mPaS, more preferred in the range of about 3 to about 7 mPaS measured at 2 wt % solution in water at 20° C.

In some embodiments the viscosity is measured by a Brookfield type RV viscometer using Brookfield spindle 2.

In some embodiments the film forming polymer is selected from the group of hypromellose 2910 (7-12% HP, 28-30% methoxy), hypromellose 2906 (4-7.5% HP, 27-30% methoxy), Hypromellose 2208 (4-12% HP, 19-24% methoxy), Hypromellose 1828 (23-32% HP, 16.5-20% methoxy), methylcellulose; and mixtures thereof.

In some embodiments the composition according to the invention further comprises a plasticizer.

In some embodiments the plasticizer is selected from the group consisting of a polyol, such as a sugar alcohol, such as propylene glycol, ethylene glycol, sorbitol, mannitol, erythritol, xylitol, or glycerol (Propane-1,2,3-triol); acetate esters of glycerol selected from the group consisting of the mono-, di-, and tri-acetates of glycerol; triethyl citrate; dibutyl sebacate; and dibutyl phthalate; and mixtures thereof.

In some embodiments the plasticizer is selected from the group consisting of glycerol, propylene glycol, ethylene glycol, sorbitol, triethyl citrate; and mixtures thereof.

In some embodiments the composition according to the invention further comprises a cationic salt.

In some embodiments the cationic salt is selected from the group consisting of calcium salt, magnesium salt; and mixtures thereof.

In some embodiments the composition according to the invention further comprises one or more components selected from the group consisting of surfactant(s) such as simethicone, sodium lauryl sulfate; coloring agent(s); flavoring agent(s); and mixtures thereof.

In some embodiments the concentration of alginate in the composition according to the invention is from about 0.2 to about 10% by weight based on the total weight of the aqueous composition.

In some embodiments the concentration of the total amount of HPMC and/or MC is from about 8 to about 25% by weight based on the total weight of the aqueous composition.

In some embodiments the alginate is present in an amount of at least about 0.2%, preferably of at least about 0.5%, more preferably of at least about 0.9% by weight based on the total weight of the aqueous composition.

In some embodiments the HPMC and/or MC is present in an in an amount of at least about 8%, preferably of at least about 10%, more preferably of at least about 14% by weight based on the total weight of the aqueous composition.

In some embodiments the water in the composition according to the invention is present in an amount of not more than about 96%, preferably not more than about 92%, more preferably not more than about 87% by weight based on the total weight of the aqueous composition.

In some embodiments the composition according to the invention has a gelation temperature (T_{gel}) of the aqueous composition in the range of 15-45° C.

In some embodiments the composition according to the invention has a viscosity in the range of about 500 to about 6000 mPa·s measured at a temperature in the range of 9° C. to 1° C. below the gelling temperature of the aqueous composition measured by a Brookfield type RV viscometer using Brookfield spindle 2.

In some embodiments the composition according to the invention is for use in providing sustained or targeted release of a pharmaceutically or nutraceutically active ingredient.

In some embodiments the composition according to the invention is for use in providing delayed or enteric release of a pharmaceutically or nutraceutically active ingredient.

This invention concerns aqueous compositions comprising alginate with low viscosity, such as low MW alginate and a low viscosity film forming polymer, such as low MW Hypromellose or hydroxypropyl methyl cellulose (HPMC), processes for producing the compositions, and coated dosage forms and capsule shells made from the aqueous compositions for delayed release and/or enteric release applications.

Many actives and/or APIs cannot be released immediately in stomach condition as they can cause irritation to stomach mucosa, degrade in stomach acid, or react with stomach acid. Various known esterified cellulose ethers and polymethacrylates have been used as enteric polymers for pharmaceutical dosage forms, such as methylcellulose phthalate (MCP), hydroxypropyl methylcellulose phthalate (HPMCP), methylcellulose succinate (MCS), or hydroxypropyl methylcellulose acetate succinate (HPMCAS). These polymers are used for coating dosage forms, such as tablets, micro-particulates or capsules.

The present inventors found a thermal gelling polymeric capsule shell that can be prepared from an aqueous composition which comprises low MW Na-alginate and low MW HPMC. These materials offer delayed/enteric release without additional gelling agent and effectively avoid additional coating steps on capsules. Additionally, the inventive blends of HPMC/Na-alginate offers similar gelation temperature but lower viscosity that will be beneficial for capsule manufacturing process. The formed capsule shells can also go through 2^{nd} dipping into hot CaCl₂) solutions to enhance film strength and enteric performance. When the molecular weight of Na-alginate is increased to higher than that of Manucol LD/LB, the film strength and enteric performance of the formulated blends will be further improved.

The gelation temperature (T_{gel}) is used herein in its standard meaning referring to the temperature at which the blend according to the invention undergoes reversable thermal gelation. The T_{gel} may be measured by methods known to the person skilled in the art. In a preferred embodiment T_{gel} is the temperature measured at minimum complex viscosity (|η*|) during heating.

The blends can also be utilized in coating, micro-encapsulations and other film-forming applications.

The advantage of this invention is that it provides an aqueous delayed release/enteric release formulations and corresponding capsule/films that avoiding multiple formulation steps and multiple coating steps.

### Alginate

By the term “alginate” is meant a linear unbranched polymer containing (1-4)-linked β-D-mannuronic acid (M) and α-L-guluronic acid (G) residues. Alginates are found in brown seaweeds (class, Phaeophyceae) and other sources. Alginates are not random copolymers, but consist of blocks of similar and alternating residues, for example, MMMM, GGGG, and GMGM, and are generally useful in the form of alginic acid or salts thereof. The alginates from Phaeophyceae seaweeds vary in the proportion of M and include Lessonia nigrescens (53-60% M), Laminaria digitata (about 59% M), Macrocystis pyrifera (about 60% M), Ecklonia maxima, and Laminaria saccharina.

NMR The term alginate also refers to alginic acid, salts, and esters thereof, such as sodium alginate, potassium alginate, and other monovalent alginate salts.

NMR An alginate that may be suitably used according to the present invention is capable of forming a low viscosity aqueous solution at room temperature at a concentration of 1% (w/v). More specifically, the alginate for use according to the invention is an alginate, which upon dissolution in water in a concentration of 1% (w/v), at a temperature of 20° C., forms an aqueous solution having a viscosity value of less than 200 mPaS. According to one embodiment of the invention, the viscosity measured for 1% (w/v) aqueous solution of the alginate is in the range between 2 and 200 mPaS. According to an alternative embodiment, the alginate selected for preparing the film is such that the viscosity of 1% (w/v) aqueous solution thereof is less than 100 mPaS, and preferably in the range between 10 to 70 mPaS at a temperature of 20° C. The viscosity measurements may be carried out on a Brookfield viscometer, spindle N° 2, at 20 rpm to 60 rpm.

NMR In some embodiments, the aqueous film-forming compositions of the present invention contains alginate having an average M content of from 25%-62% by weight of the M and G content. In some embodiments, the average M content of the alginate, by weight of the M and G content, can be at least about 25%, 30%, 40%, 50%, 55%, 56%, and not more than about 50%, 55%, 56%, 57%, 58%, 59%, 60%, 61% or 62%. The M content may also be in the range of from 53% to 59% by weight of the M and G content.

NMR Alginate used according to the present invention is typically obtained from a variety of alginate producing seaweeds. The M content in seaweeds can vary depending on their life cycle at harvest, plant specificity, etc. Seaweeds generally considered to produce alginate having an M content suitable for present invention include Lessonia nigrescens, Laminaria digitata, Macrocystis pyrifera, Ecklonia maxima and Laminaria saccharina. It is also within the scope of the present invention to use a blend of alginates having various M content provided the M content of all alginates in the blend have an average M content of 25-62% M. The average M content in alginate is typically determined by ¹H—. The alginate can typically be extracted from the seaweed using, for example, standard commercial extraction processes such as an aqueous process including an acidic pretreatment followed by an alkaline extraction. Such conventional techniques are incorporated herein by reference.

NMR The extracted alginates of the present invention having an M content of 25-62% M may have a high molecular weight and, thus, a high viscosity such as 200-1,000 cps in a 1% solution. Thus, the alginate may have to be degraded to obtain a suitable average viscosity. The degradation is typically done by conventional heat treatment processes of the alginic acid. These conventional processes are incorporated herein by reference.

Additional viscosity tests that may be used for identifying preferred alginates to be practiced according to the present invention may be carried out at different concentrations of the alginate and/or different temperatures.

Examples of suitable commercial grades of alginic acid include (viscosity of a 1% solution):
Alginic acid HFD2 (Alginate Industries, U.K.) which typically has a viscosity of 15-25 centipoises (cps),
Protacid F 120 (Protan & Fagertun, Norway) which typically has a viscosity of 15-25 cps,
Kelacid (Kelco, U.S.A.) which typically has a viscosity of 10-20 cps, and
Algocean (Sobalg, France) which typically has a viscosity of 15-25 cps.

Examples of alternative suitable commercial grades of sodium alginate include:
Manucol LD (IFF (formerly FMC BIOPOLYMER)) which typically has a viscosity of 10 cps,
Manucol LB (IFF (formerly FMC BIOPOLYMER)) which typically has a viscosity of 20-100 cps (3% solution),
Manucol LF (IFF (formerly FMC BIOPOLYMER)) which typically has a viscosity of 20 cps,
Protanal LF 5/60 or LF 5/40 (Protan & Fagertun) both of which typically have a viscosity of 10 cps, and Protanal LF 5/120 M which typically has a viscosity of 20 cps.
Manucol LHF and Protanal LF 10/120 which typically has a viscosity of 40 cps.

An example of a partially neutralised alginic acid is one coded PE III (Protan & Fagertun) which is approximately 50% neutralised and has typically a viscosity of 15 cps.

Various blends may also be used such as blends of alginic acid or other grades of sodium alginate, such as for example a 40/60 blend of Manucol LF with Manucol LHF which has a viscosity of 24 cps.

### Film Forming Polymers

Any suitable film forming polymer may be used according to the present invention if the viscosity is in the range of about 2 to about 20 mPaS measured at 2 wt % solution in water at 20° C. The person skilled in the art will know these suitable polymers.

Suitable film forming polymer used according to the present invention includes low-viscosity hydroxypropylcellulose, hydroxypropyl methylcellulose (HPMC) and methylcellulose (MC), hypromellose 2910 (7-12% HP, 28-30% methoxy), hypromellose 2906 (4-7.5% HP, 27-30% methoxy), Hypromellose 2208 (4-12% HP, 19-24% methoxy), Hypromellose 1828 (23-32% HP, 16.5-20% methoxy). Commercially available methylcelluloses and hydroxypropyl methylcelluloses include METHOCEL (trademark) series from IFF, Japanese Pharmacopoeia METOLOSE (trademark) series and METOLOSE series for food additives from Shin-Etsu Chemical Co., Ltd., AnyCoat-C or AnyAddy (trademark) series from Lotte (former Samsung) Fine Chemicals Co., Ltd., and Benecel (trademark) series from Ashland.

In the present disclosure, it is preferred to use film forming polymers having a “viscosity” of 2 mPa·s or more when in the form of a 2% by mass aqueous solution at 20° C. The “viscosity” can be measured in accordance with the section of methylcellulose and hypromellose, which is formulated based on the International Harmonization Plan in and after the 15th Pharmacopoeia. In other words, the “viscosity” refers to the value of viscosity (mPa·s) of a 2% by mass aqueous solution of the water-soluble cellulose at 20° C.±0.1° C.

Also, the viscosity labeled by the compound manufacturer (sometimes referred to as “viscosity grade value”) may be employed as the “viscosity”. Regarding the labeled viscosity and the range of the labeled viscosity, for example, METHOCEL (trademark) series from IFF or METOLOSE (tradename) series from Shin-Etsu Chemical Co., Ltd, are described to have a viscosity value that is 80 to 120% of the labeled viscosity when the labeled viscosity is less than 600 mPas and a viscosity value that is 75 to 140% of the labeled viscosity when the labeled viscosity is 600 mPa·s or more. With regard to the lower limit value of 2 mPa·s in the present invention, the labeled viscosity can be directly used as the “viscosity” as long as the intent of the present invention is not impaired.

In the present disclosure, a preferred lower limit value of the viscosity is 2 mPa·s, such as 4, 6, 8 or 10 mPa·s. A preferred upper limit value of the viscosity is 20 mPas, such as 18, 16, 14, 12, or 10 mPa·s.

### Plasticizer

Examples of the plasticizer include surfactants such as sucrose fatty acid ester, glycerin fatty acid ester, monoglycerin fatty acid ester, polyoxyethylene sorbitan fatty acid ester and the like, ester of citric acid such as triethyl citrate (TEC); polyhydric alcohol such as glycerin, propylene glycol, polyethylene glycol, etc., glucose, Sugar such as fructose and glucose liquid sugar, sugar such as sucrose, sugar alcohol such as sorbitol, maltitol, mannitol, erythritol, xylitol, dodecanol, tridecanol, tetradecanol, pentadecanol, hexadecanol, heptadecanol, octadecanol, Higher alcohols such as hexadecyl alcohol, isostearyl alcohol, 2-octyldodecanol and the like (preferably having 6 to 22 carbon atoms), and oils and fats such as medium chain fatty acid esters (preferably having 6 to 12 carbon atoms) It is below. These may be used alone or in combination of two or more.

By the term “capsule” is meant an enclosing structure having a content (fill material) and surface (film). When substantially dry, the film constrains the fill material. Preferably, the film disintegrates under conditions found in the mammalian small intestine. A finished capsule is substantially dry after exposure to an excess of gas at a relative humidity of 30% or less at room temperature for a day, or the equivalent. Drying means substantially drying. A capsule is “wet” when not substantially dry or when substantially in contact with an aqueous solution.

### EXPERIMENTAL SECTION

### Example 1

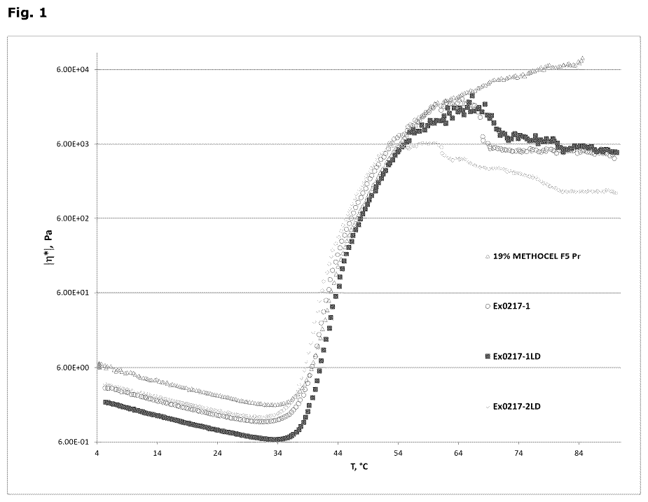

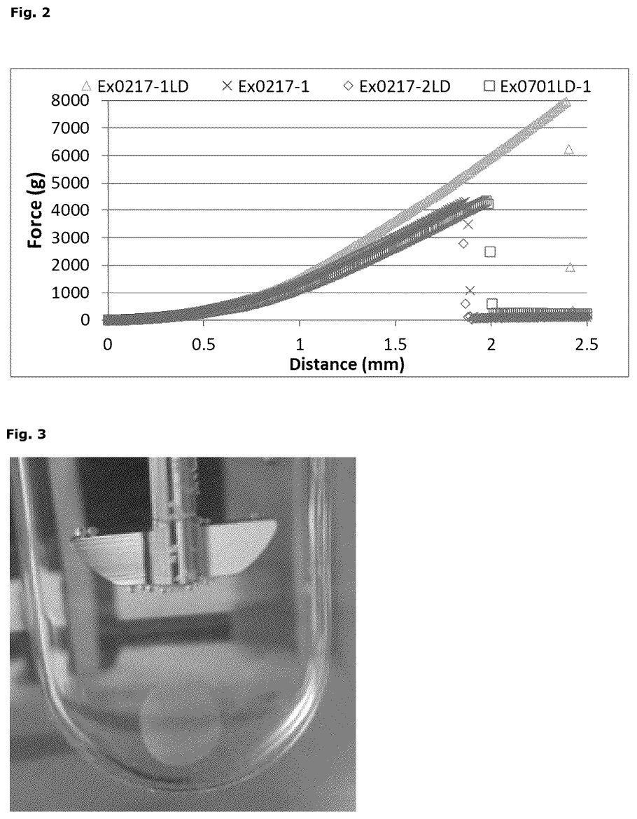

Examples of formulations with alginate are shown in tables 1, 2 and 3. Rheology of inventive formulations of those listed in Table 1 and 2 are shown in FIG. 1. The viscosity of inventive formulations showed lower viscosity than that of the commercial HPMC capsule materials. Additionally, the inventive formulations showed similar gelation temperature and gel strength as that of the commercial HPMC capsule materials. All film samples, Ex0217-1LD, Ex0217-2LD, Ex0217-1, Ex0701LD-1 showed good film strength as shown in FIG. 2.

The delay and enteric performance of the film were carried out using the USP dissolution Apparatus 2 with 100 mL vessels and mini paddles.
Equipment configuration: Modified USPII—with 1000 mL vessels and paddles
Dissolution media: 100 ml degassed pH 1.2 HCl buffer
Temperature: 37±0.5° C.
Stir speed: 50 RPM

All film samples, Ex0217-1LD, Ex0217-2LD, Ex0217-1, Ex0701LD-1 appeared intact after 2 hours in pH 1.2 dissolution media as shown in FIG. 3.

TABLE 1
Delayed release formulation of HPMC and Na-alginate blends.

| Samples | Ex0217-1LD, fraction | Ex0217-2LD, fraction |
|---|---|---|
| METHOCEL ™ F4 Pr | 0.15 | 0.15 |
| Na-alginate | 0.01 | 0.02 |
| (Manucol LD) | | |
| TEC | 0.004 | 0.008 |
| Deionized water | 0.836 | 0.822 |
| Total | 1.0000 | 1.0000 |

TABLE 2
Delayed release formulation of HPMC and Na-alginate blends.

| Samples | Ex0217-1, fraction |
|---|---|
| METHOCEL ™ F4 Pr | 0.15 |
| Manucol LB | 0.01 |
| TEC | 0.004 |
| Deionized water | 0.836 |
| Total | 1.0000 |

TABLE 3
Delayed release formulation of HPMC and Na-alginate blends.

| Samples | Ex0701LD-1, fraction |
|---|---|
| METHOCEL ™ F4 Pr | 0.16 |
| Manucol LD | 0.012 |
| CaCl2 | 0.0005 |
| TEC | 0.0048 |
| Deionized water | 0.818 |
| Total | 1.0000 |

1. An aqueous film-forming composition comprising a) alginate having a viscosity in the range of about 2 to about 200 mPaS measured at 1 wt % solution in water at 20° C. and b) a film forming polymer having a viscosity in the range of about 2 to about 20 mPaS measured at 2 wt % solution in water at 20° C.

2. The composition according to claim 1, wherein the alginate is selected from the group consisting of sodium alginate, potassium alginate, other monovalent alginate salts, and mixtures thereof.

3. The composition according to claim 1, wherein the alginate is sodium alginate.

4. The composition according to claim 1, wherein the alginate has a viscosity in the range of about 2 to about 30 mPaS measured at 1 wt % solution in water at 20° C.

5. The composition according to claim 1, wherein the film forming polymer is selected from the group consisting of hydroxypropyl methylcellulose (HPMC), carboxymethyl cellulose (CMC) and methylcellulose (MC), hydroxypropyl cellulose (HPC), and mixtures thereof.

6. The composition according to claim 1, wherein the film forming polymer has a viscosity in the range of about 3 to about 7 mPaS measured at 2 wt % solution in water at 20° C.

7. (canceled)

8. The composition according to claim 1, wherein the film forming polymer is selected from the group of hypromellose 2910 (7-12% HP, 28-30% methoxy), hypromellose 2906 (4-7.5% HP, 27-30% methoxy), Hypromellose 2208 (4-12% HP, 19-24% methoxy), Hypromellose 1828 (23-32% HP, 16.5-20% methoxy), methylcellulose, and mixtures thereof.

9. The composition according to claim 1, further comprising a plasticizer.

10. The composition according to claim 9, wherein the plasticizer is selected from the group consisting of a polyol; acetate esters of glycerol selected from the group consisting of the mono-, di-, and tri-acetates of glycerol; triethyl citrate; dibutyl sebacate; dibutyl phthalate; and mixtures thereof.

11. (canceled)

12. The composition according to claim 1, further comprising a cationic salt.

13. The composition according to claim 12, wherein the cationic salt is selected from the group consisting of calcium salt, magnesium salt, and mixtures thereof.

14. (canceled)

15. The composition according to claim 1, wherein the concentration of alginate is from about 0.2 to about 10% by weight based on the total weight of the aqueous composition.

16. The composition according to claim 1, wherein;
the film forming polymer is HPMC and/or MC, and
the concentration of the total amount of HPMC and/or MC in the aqueous composition is from about 8 to about 25% by weight based on the total weight of the aqueous composition.

17-19. (canceled)

20. The composition according to claim 1, which composition has a gelation temperature (T_{gel}) of the aqueous composition in the range of 15-45° C.

21. The composition according to claim 1, which composition has a viscosity in the range of about 500 to about 6000 mPa·s measured at a temperature in the range of 9° C. to 1° C. below the gelling temperature of the aqueous composition measured by a Brookfield type RV viscometer using Brookfield spindle 2.

22-24. (canceled)

25. A capsule film made from the composition according to claim 1.

26. A capsule shell comprising a) alginate having a viscosity in the range of about 2 to about 200 mPaS measured at 1 wt % solution in water at 20° C. and b) a film forming polymer having a viscosity in the range of about 2 to about 20 mPaS measured at 2 wt % solution in water at 20° C.

27-28. (canceled)

29. A capsule comprising the capsule shell according to claim 26.

30. The capsule of claim 29 further comprising a pharmaceutically active ingredient, dietary supplement, flavouring agent, foodstuff, agrochemical or scent.

31. (canceled)

32. A process for producing capsule film, capsule shell, or capsule, wherein the process comprises:
providing the aqueous composition according to claim 1,
pre-heating moulding pins to a temperature at highest viscosity point ±5° C. of the aqueous composition above the gelation temperature of the aqueous composition,
dipping the pre-heated moulding pins into the aqueous composition at a temperature below gelation temperature of the aqueous composition,
forming a film on said moulding pins by withdrawing said pins from the aqueous composition, and
drying the film on the moulding pins above the gelation temperature of the aqueous composition.

33. (canceled)
